(19) United States
(12) Patent Application Publication
(10) Pub. No.: US 2020/0305982 A1
(43) Pub. Date: Oct. 1, 2020

(54) APPARATUS AND METHOD FOR MEASURING DEPTH OF BIT IN ENT DRILL

(71) Applicant: Acclarent, Inc., Irvine, CA (US)

(72) Inventors: Fatemeh Akbarian, Rancho Palos Verdes, CA (US); Babak Ebrahimi, Irvine, CA (US); Itzhak Fang, Irvine, CA (US); Jetmir Palushi, Irvine, CA (US); Henry F. Salazar, Pico Rivera, CA (US); Ehsan Shameli, Irvine, CA (US)

(21) Appl. No.: 16/744,595

(22) Filed: Jan. 16, 2020

Related U.S. Application Data
(60) Provisional application No. 62/825,851, filed on Mar. 29, 2019.

(51) Int. Cl. A61B 34/20; A61B 17/16

(52) U.S. Cl. CPC A61B 34/20; A61B 2017/00424; A61B 17/1688

(57) ABSTRACT

A surgical system and method for determining insertion depth of a cutting member includes a body assembly, a securement mechanism, a base position sensor, and a cutting member sensor. The body assembly defines a longitudinal axis and is configured to receive the cutting member. The securement mechanism is operatively connected to the body assembly and configured to releasably secure the cutting member relative to the body assembly. The base position sensor is configured to be detected as a base position by a navigation system. The cutting member sensor is positioned a predetermined longitudinal sensor distance from the base position sensor and configured to detect an adjustable longitudinal position of the cutting member. The sensors are configured to communicate with the navigation system for determining a position of the distal reference feature within an anatomical passageway of a patient in any selected adjustable longitudinal position of the cutting member.

### PRIORITY

This application claims priority to U.S. Provisional Patent App. No. 62/825,851, entitled “Apparatus and Method for Measuring Depth of Bit in ENT Drill,” filed Mar. 29, 2019, the disclosure of which is incorporated by reference herein.

### BACKGROUND

Surgical instruments, such as surgical drilling instruments, contain features that facilitate operation within or adjacent to an anatomical passageway of a patient, such as during incisions of mucosa, removal of bone, or dilation of an anatomical passageway. Anatomical passageways that may undergo such operations may include ostia of paranasal sinuses (e.g., to treat sinusitis), the larynx, the Eustachian tube, or other passageways within the ear, nose, or throat, etc. In addition to the above described operations, or similar operations, it may be desirable to remove tissue within or adjacent to an anatomical passageway before, during, or after the above described operations. One method of removing tissue within or adjacent to an anatomical passageway of the patient involves obtaining a surgical drilling instrument with a body assembly having a cutting member that extends distally from the body assembly. An operator may then insert a distal end of the cutting member having a cutting feature within the nostril or mouth of the patient toward a desired location within the patient. With the distal end of the cutting member inserted within the patient, the operator may manipulate the surgical drilling instrument to remove extraneous and/or undesired matter within or adjacent to an anatomical passageway of the patient.

Surgical instruments may be located within the patient in some instances with cooperative use of an image-guided surgery (IGS) system. IGS is a technique where a computer is used to obtain a real time correlation of the location of the surgical instrument that has been inserted into the patient's body to a set of preoperatively obtained images (e.g., a CT or MRI scan, 3-D map, etc.) so as to superimpose the current location of the instrument on the preoperatively obtained images. In some IGS procedures, a digital tomographic scan (e.g., CT or MM, 3-D map, etc.) of the operative field is obtained prior to surgery. A specially programmed computer is then used to convert the digital tomographic scan data into a digital map. During surgery, operators use surgical instruments having sensors (e.g., electromagnetic coils that emit electromagnetic fields and/or are responsive to externally generated electromagnetic fields) mounted on the surgical instruments to perform surgical procedures while the sensors send data to the computer indicating the current position of the sensors and, in turn, the surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot, etc.) showing the real time position of the surgical instrument relative to the anatomical structures shown in the scan images. More particularly, these tomographic scan images show the real time position of one or more portions of the surgical instrument fixed relative to the sensors. In this manner, the operator is able to know the precise position of such fixed portions of each sensor-equipped instrument by viewing the video monitor even if the operator is unable to directly visualize the instrument itself at its current location within the body.

When applied to functional endoscopic sinus surgery (FESS), balloon sinuplasty, and/or other ENT procedures, the use of IGS systems allows the operator to achieve more precise movement and positioning of the surgical instruments than can be achieved by viewing through an endoscope alone because a typical endoscopic image is a spatially limited, 2-dimensional, line-of-sight view. The use of IGS systems provides a real time, 3-dimensional view of all of the anatomy surrounding the operative field, not just that which is actually visible in the spatially limited, 2-dimensional, direct line-of-sight endoscopic view. As a result, IGS may be particularly useful during performance of FESS, balloon sinuplasty, and/or other ENT procedures where normal anatomical landmarks are not present or are difficult to visualize endoscopically.

It may be desirable to efficiently use surgical instruments that contain sensors for use with navigation systems, such as IGS systems, particularly for knowing a precise position of one or more portions of a surgical instrument that may be adjustable relative to such fixed portions discussed above. While several different methods to use surgical instruments configured for removal of tissue from the body, it is believed that no one prior to the inventors has used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic view of an exemplary surgical system having a navigation system and a surgical drilling instrument being used on a patient seated in an exemplary medical procedure chair;

FIG. 2 depicts a perspective view of the surgical drilling instrument of FIG. 1 having a body assembly fitted with a cutting member;

FIG. 3A depicts an enlarged, schematic, side view of the surgical drilling instrument and the navigation system of FIG. 1 with a cutting member in an extended state and having various portions hidden for additional clarity;

FIG. 3B depicts the enlarged, schematic, side view of the surgical drilling instrument similar to FIG. 3A, but showing the cutting member proximally translating from the extended state toward a shortened state; and

FIG. 3C depicts the enlarged, schematic, side view of the surgical drilling instrument similar to FIG. 3B, but showing the cutting member in the shortened state.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handle of a body assembly. Thus, an end effector, such as a cutting member, is distal with respect to the more proximal handle. It will be further appreciated that, for convenience and clarity, spatial terms such as “coaxial,” and “longitudinal” also are used herein for reference to relative positions and directions. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Image Guided Surgery Navigation System

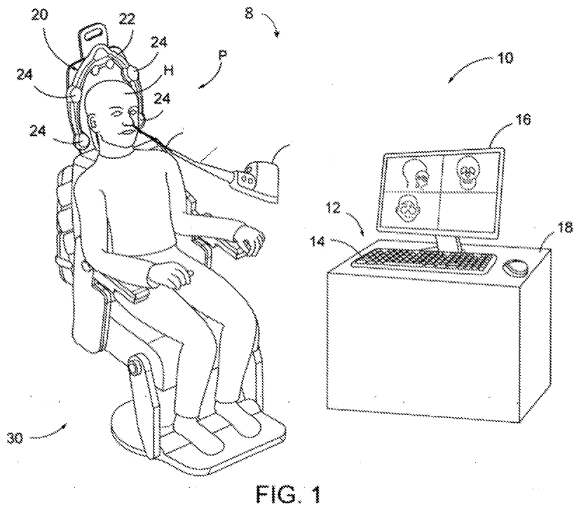

FIG. 1 shows an exemplary surgical system (8) having an IGS navigation system (10) enabling an ENT procedure to be performed using image guidance with a surgical instrument, such as a surgical drilling instrument (100). When performing a medical procedure within a head (H) of a patient (P), it may be desirable to have information regarding the position of an instrument within the head (H) of the patient (P), particularly when the instrument is in a location where it is difficult or impossible to obtain an endoscopic view of a working element of the instrument within the head (H) of the patient (P). In addition to or in lieu of having the components and operability described herein, IGS navigation system (10) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; and U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein.

IGS navigation system (10) of the present example comprises a field generator assembly (20), which comprises a set of magnetic field generators (24) that are integrated into a horseshoe-shaped frame (22). Field generators (24) are operable to generate alternating magnetic fields of different frequencies around the head (H) of the patient (P). One or more portions of surgical drilling instrument (100), such as a cutting member (106) and a sleeve (116), are inserted into the head (H) of the patient (P) in this example. Frame (22) is mounted to a chair (30) of surgical system (8), with the patient (P) being seated in chair (30) such that frame (22) is located adjacent to the head (H) of the patient (P). By way of example only, chair (30) and/or field generator assembly (20) may be configured and operable in accordance with at least some of the teachings of U.S. Pat. Pub. No. 2018/0310886, entitled “Apparatus to Secure Field Generating Device to Chair,” published Nov. 1, 2018, the disclosure of which is incorporated by reference herein.

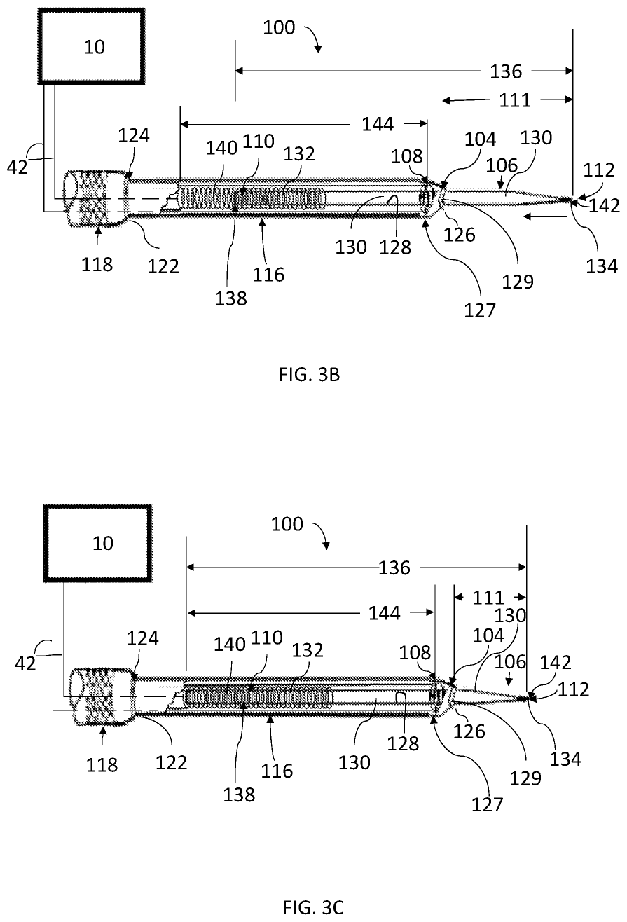

IGS navigation system (10) of the present example further comprises a processor (12), which controls field generators (24) and other elements of IGS navigation system (10). For instance, processor (12) is operable to drive field generators (24) to generate alternating electromagnetic fields and process signals from sleeve (116) to determine the location of a base position sensor (108) (see FIG. 3A) in sleeve (40) within the head (H) of the patient (P). Processor (12) comprises a processing unit (not shown) communicating with one or more memories (not shown). Processor (12) of the present example is mounted in a console (18), which comprises operating controls (14) that include a keypad and/or a pointing device such as a mouse or trackball. A physician uses operating controls (14) to interact with processor (12) while performing the surgical procedure.

Sleeve (116) includes base position sensor (108) (see FIG. 3A) that is responsive to positioning within the alternating magnetic fields generated by field generators (24). A coupling unit (42) is secured to the proximal end of surgical drilling instrument (100) and is configured to provide communication of data and other signals between console (18) and sleeve (116). Coupling unit (42) may provide wired or wireless communication of data and other signals.

In the present example, base position sensor (108) (see FIG. 3A) of sleeve (116) comprises at least one coil as discussed below in greater detail. When such a coil is positioned within an alternating electromagnetic field generated by field generators (24), the alternating magnetic field may generate electrical current in the coil, and this electrical current may be communicated along the electrical conduit(s) in surgical drilling instrument (100) and further to processor (12) via coupling unit (42). This phenomenon enables IGS navigation system (10) to determine the location of a portion of surgical drilling instrument (100) containing base position sensor (108) (see FIG. 3A) or other medical instrument (e.g., dilation instrument, surgical cutting instrument, etc.) within a three-dimensional space (i.e., within the head (H) of the patient (P), etc.). To accomplish this, processor (12) executes an algorithm to calculate location coordinates of a fixed portion of surgical drilling instrument (100) from the position related signals of the coil(s) of base position sensor in sleeve (116). Any such portion of surgical drilling instrument (100) that is fixed relative to base position sensor (108) (see FIG. 3A) may be calculated with known, fixed location coordinates. While base position sensor (108) (see FIG. 3A) is located in sleeve (116) in this example and as discussed below, such a position sensor may be integrated into other portions of surgical drilling instrument (100) as well as various other kinds of instruments.

Processor (12) uses software stored in a memory of processor (12) to calibrate and operate IGS navigation system (10). Such operation includes driving field generators (24), processing data from surgical drilling instrument (100), processing data from operating controls (14), and driving display screen (16). In some implementations, operation may also include monitoring and enforcement of one or more safety features or functions of IGS navigation system (10). Processor (12) is further operable to provide video in real time via display screen (16), showing the position of sleeve (116) of surgical drilling instrument (100) in relation to a video camera image of the patient's head (H), a CT scan image of the patient's head (H), and/or a computer-generated three-dimensional model of the anatomy within and adjacent to the patient's nasal cavity. Display screen (16) may display such images simultaneously and/or superimposed on each other during the surgical procedure. Such displayed images may also include graphical representations of instruments that are inserted in the patient's head (H) such that an operator may view the virtual rendering of surgical drilling instrument (100) at its actual location in real time. By way of example only, display screen (16) may provide images in accordance with at least some of the teachings of U.S. Pat. Pub. No. 2016/0008083, entitled “Guidewire Navigation for Sinuplasty,” published Jan. 14, 2016, now U.S. Pat. No. 10,463,242, issued Nov. 5, 2019, the disclosure of which is incorporated by reference herein. In the event that the operator is also using an endoscope, the endoscopic image may also be provided on display screen (16).

The images provided through display screen (16) may help guide the operator in maneuvering and otherwise manipulating instruments within the patient's head (H) when such instruments incorporate sleeve (116) or other portions of surgical drilling instrument (100) that contain base position sensor (108) (see FIG. 3A). It should also be understood that other components of a surgical instrument and other kinds of surgical instruments, as described below, may incorporate a sensor like base position sensor (108) (see FIG. 3A) of surgical drilling instrument (100).

II. Surgical Drilling Instrument with Sensors for Imaging an Adjustable Cutting Member

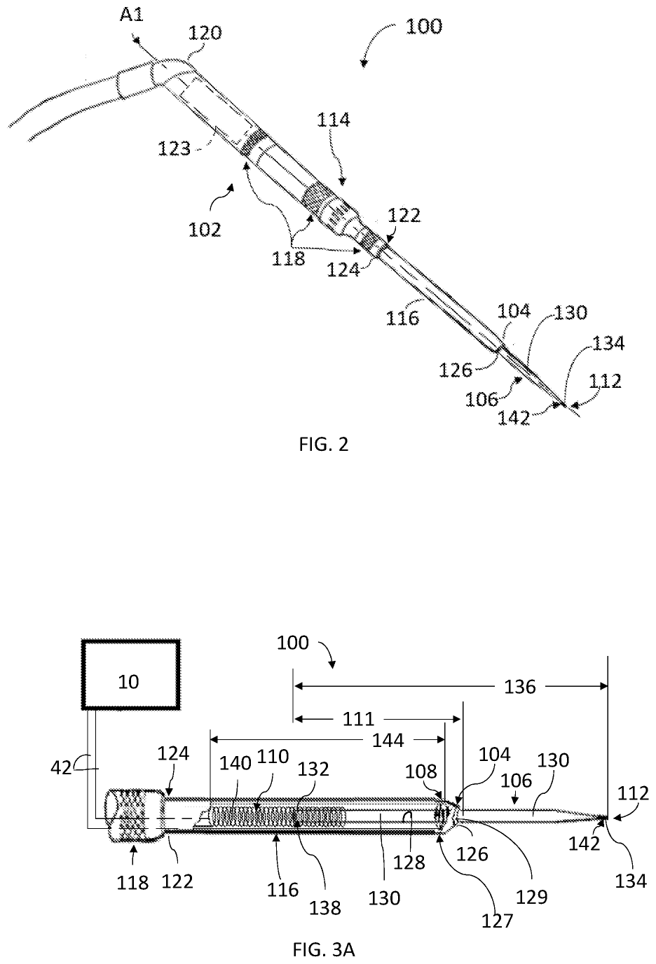

FIGS. 2-3A show surgical drilling instrument (100) with sleeve (116) and cutting member (106) as briefly discussed above for removing tissue, such as bone tissue, from the nasal cavity, as well as from any other suitable location. Surgical drilling instrument (100) of the present example also includes a body assembly (102), a securement mechanism (104), base position sensor (108), and a cutting member sensor (110). Body assembly (102) coaxially receives cutting member (106) through securement mechanism (104) to releasably secure cutting member (106) at one of various insertion depths, such as one example of an insertion depth (111). The operator may selectively adjust cutting member (106) to any desired insertion depth (111) relative to sleeve (116), while cutting member sensor (110) detects the adjusted insertion depth (111) as discussed below in greater detail. Navigation system (10) thus communicates with base position sensor (108) to locate base position sensor (108) within the head (H) (see FIG. 1) of the patient (P) (see FIG. 1) and then, based on the detected insertion depth (111) of cutting member (106), determines a particular position of a distal reference feature (134) on cutting member (106) within the head (H) (see FIG. 1) of the patient (P) (see FIG. 1). Thereby, navigation system (10) and surgical drilling instrument (100) are collectively configured to determine the particular position of distal reference feature (134) on cutting member (106) in any anatomical passageway of the patient (P) (see FIG. 1) and in any selected adjustable longitudinal position of cutting member (106). Moreover, such determination is made by navigation system (10) without a position sensor secured on or fixed relative to cutting member (106).

In the present example, body assembly (102) has a handle (114) with sleeve (116) rigidly and distally extending therefrom along a longitudinal axis (A1) for insertion into the anatomical passageway. More particularly, sleeve (116) is configured to be inserted into the paranasal sinus, while handle (114) is configured to be gripped by the operator during use, such by gripping knurled portion (118). Handle (114) distally extends along the longitudinal axis (A1) from a proximal handle end (120) to a distal handle end (122). Handle (114) contains a motorized drive assembly (123) operatively connected to cutting member (106) via securement mechanism (104) for driving rotation of cutting member (106). An alternative drive assembly, such as a pneumatic drive assembly, may similarly connect to cutting member (106) for driving rotation of cutting member (106). Handle (114) may further include controls (not shown) for the operation of surgical drilling instrument (100) or the controls may be located remotely. Sleeve (116) also extends distally along the longitudinal axis (A1) from a proximal sleeve end (124) to a distal sleeve end (126) and defines a hollow (128) configured to receive cutting member (106).

To this end, securement mechanism (104) of the present example includes a collet (129) that releasably engages with a portion of cutting member (106) to longitudinally retain cutting member (106) within sleeve (116) while still allowing cutting member (106) to be rotatably driven for use. The operator rotatably loosens and tightens collet (129) of securement mechanism (104) to respectively disengage from cutting member (106) in an unlocked state and engage cutting member (106) in a locked state. While cutting member (106) is longitudinally fixed relative to sleeve (116) in the locked state, collet (129) in the unlocked state is loosened from cutting member (106) to allow the operator to translatably slide cutting member (106) relative to sleeve (116) and adjust insertion depth (111) of cutting member (106). While securement mechanism (104) includes collet (129) for selectively securing cutting member (106) relative to sleeve (116), alternative securement mechanism (not shown) may include a chuck, fastener, pin, magnet or other coupling to longitudinally secure cutting member (106) relative to sleeve (116). The invention is thus not intended to be unnecessarily limited to the particular securement mechanism (104) shown and described herein.

Cutting member (106) of the present example is coaxially disposed in sleeve (116) and distally extends from distal sleeve end (126). Cutting member (106) more particularly has a shaft body (130) distally extending from a proximal shaft end (132) to a distal tip (112) as well as a cutting feature (142) positioned on shaft body (130). Cutting feature (142) includes a plurality of flutes (not shown) with sharpened edges (not shown) proximally spiraling from distal tip (112) about shaft body (130) in the present example and may also be referred to as a “drill bit.” Such cutting feature (142) is configured to bore a hole through tissue for removal from the anatomical passageway. It will be appreciated that alternative cutting feature arrangements may be alternatively structured and/or positioned for removing tissue, and the invention is not intended to be unnecessarily limited to the particular cutting member (106) shown and described herein.

As discussed briefly above, cutting member (106) has distal reference feature (134), which, in the in the present example, is distal tip (112) of cutting member (106). Navigation system (10) thus indicates the particular position of distal tip (112) as distal reference feature (134) on cutting member (106) although any other feature or position on cutting member (106) may be an alternative distal reference feature (134) in another example. Distal tip (112) as distal reference feature (134) is positioned at a fixed, predetermined reference distance (136) from a proximal reference feature (138) along longitudinal axis (A1). Proximal reference feature (138), which is proximal shaft end (132) in the present example, is configured to be sensed by cutting member sensor (110) for detecting the longitudinal position of proximal shaft end (132) along longitudinal axis (A1). Based on the detected longitudinal position of proximal shaft end (132) as proximal reference feature (138) as well as predetermined reference distance (136) to distal tip (112) as distal reference feature (134), navigation system (10) determines the adjusted insertion depth (111) of cutting member (106) relative to sleeve (116).

Cutting member sensor (110) shown in FIG. 3A more particularly includes a magnetic coil (140) that receives a sinusoidal voltage or current, such as from navigation system (10), and senses coil inductance, which changes measured inductance values by longitudinally repositioning proximal shaft end (132) through magnetic coil (140). Each particular inductance value among a range of possible inductance values thus respectively indicates a particular adjusted insertion depth (111) selected by the operator. While proximal reference feature (138) is proximal shaft end (132) in the present example, any such feature fixed some predetermined reference distance from the desired distal reference feature (134) may be similarly used. For example, proximal reference feature (138) may alternatively be a magnet and/or a dissimilar metal fixed to shaft body (130) and sensed by cutting member sensor (110).

Both base position sensor (108) and cutting member sensor (110) are secured to sleeve (116) as shown in FIG. 3A of the present example. Base position sensor (108) is secured a predetermined longitudinal sensor distance (144), which is a fixed distance, from cutting member sensor (110) and distally positioned relative to cutting member sensor (110). More particularly, base position sensor (108) is secured on a distal sleeve end portion (127) proximate to distal sleeve end (126). However, in other examples, base position sensor (108) may be alternatively positioned so long as navigation system (10) senses the position of base position sensor (108) relative to the anatomical passageway. In this respect, base position sensor (108) is in communication with navigation system (10), while cutting member sensor (110) senses the position of the proximal reference feature (138) for communication to navigation system (10) and the determination of the adjusted insertion depth (111) of cutting member (106). Navigation system (10) and surgical drilling instrument (100) are thereby configured to determine the position of the distal reference feature (134) relative to the anatomical passageway based on the detected position of the base position sensor (108), the detected position of the proximal reference feature (138), the predetermined reference distance (136), and the predetermined longitudinal sensor distance (144). The position of distal reference feature (134), such as distal tip (112), may then be accurately displayed to the operator in real time regardless of the adjusted longitudinal position of cutting member (106) as selected by the operator.

In use, with respect to FIG. 1 and FIGS. 3A-3C, the operator views the location of the extraneous and/or undesired matter displayed on display screen (16) and determines the desired insertion depth (111) of cutting member (106) relative to an initial insertion depth (111). In the event that the desired insertion depth (111) is different than initial insertion depth (111), the operator adjusts insertion depth (111) by rotatably loosening the securement mechanism (104) to selectively unlock the cutting member (106) relative to sleeve (116) from the locked state to the unlocked state. The operator thereby translates cutting member (106) along longitudinal axis (A1) to the desired insertion depth (111) based on the location of the extraneous and/or undesired matter by either proximally or distally translating cutting member (106) relative to body assembly (102). For example, if the operator desires to remove relatively distal extraneous and/or undesired matter from the anatomical passageway of the patient (P), the operator will distally translate cutting member (106) toward an extended state, as shown in FIG. 3A. If the operator desires to remove relatively proximal extraneous and/or undesired matter from the anatomical passageway of the patient (P), the operator proximally translates the cutting member (106) toward a shortened state, as shown in FIG. 3C. In one example shown respectively in FIGS. 3A-3C, the operator translates cutting member (106) from the extended state, through various intermediate states, and to the shortened state. Once the operator selects the desired insertion depth (111), the operator rotatably tightens securement mechanism (104) to selectively lock securement mechanism (104) in the locked state. The operator may then insert distal tip (112) of surgical drilling instrument (100) into the anatomical passageway followed by a remaining portion of cutting member (106) and sleeve (116) as desired for accessing the desired anatomy about the anatomical passageway.

While positioned within the head (H) of the patient (P), screen (16) displays distal reference feature (134), such as distal tip (112), relative to the anatomical passageway also displayed on screen (16) for communication to the operator. With respect to FIG. 1 and FIG. 3A, processor (12) determines the position of adjusted distal tip (112) relative to the anatomical passageway also displayed on screen (16) based on at least the detected positions of base position sensor (108), adjusted insertion depth detected by cutting member sensor (110), and a reference table contained in memory (not shown) of processor (12). In the present example, reference table includes a plurality of discrete inductance measurements that respectively correlate to a plurality of insertion depths (111) along longitudinal axis (A1). It will be appreciated that alternative measurements may be similarly used depending on the particular cutting member sensor (110) such that the invention is not intended to be unnecessarily limited to the particular reference table described herein.

To this end, IGS navigation system (10) determines the position of base position sensor (108) as discussed above, whereas cutting member sensor (110) senses proximal shaft end (132) as proximal reference feature (138) at the desired insertion depth (111) relative to sleeve (116). This sensing of proximal shaft end (132) is more particularly the measured inductance detected by magnetic coil (140). The measured inductance is then communicated to processor (12), which compares the measured inductance to the plurality of discrete inductance measurements in the reference table. Processor (12) identifies a particular discrete inductance measurement from the reference table closest in value to the measured inductance from cutting member sensor (110) and thereby identifies a particular insertion depth (111) correlated therewith from the reference table. Processor (12) then determines the position of distal tip (112) as distal reference feature (134) based on insertion depth (111) of distal tip (112) relative to the detected position of base position sensor (108) in anatomical passageway. Finally, processor (12) directs display screen (16) to indicate the particular position of distal tip (112) as distal reference feature (134) on display screen (16) for communication to the operator. The operator may then continue with the surgical procedure as desired, such as by positioning the displayed distal tip (134) directly on the displayed desired anatomy.

The images provided through display screen (16) may help guide the operator in maneuvering and otherwise manipulating instruments within the head (H) of the patient (P). The operator operates controls (not shown) to engage drive assembly (123). Drive assembly (123) rotatably drives cutting member (106). The operator references display screen (16) which displays in real time the undesired matter and the distal reference feature (134). The operator places distal reference feature (134) into the undesired matter, which enables cutting feature (142) to remove the undesired matter from the anatomical passageway of the patient.

As discussed above, the reference table is stored on memory (not shown) of processor (12) and includes correlations of discrete inductance measurements to discrete insertion depths (111) along longitudinal axis (A1) of cutting member (106). Such correlations may be developed experimentally and/or calculated based on known measurements along longitudinal axis (A1) for inclusion in the reference table and ran as an algorithm by processor (12). In one exemplary calculation, detection of base position sensor (108) via navigation system (10) identifies a base position coordinate from which to calculate distal reference feature (134), such as distal tip (112). More particularly, from base position coordinate, subtracting predetermined longitudinal sensor distance (144) calculates a cutting member sensor coordinate from base position coordinate. In turn, the detected proximal reference feature (138), such as a proximal shaft end (132), provides a proximal reference coordinate from the cutting member sensor coordinate. Finally, simply add the predetermined reference distance from the proximal reference coordinate to calculate a distal reference coordinate for distal reference feature (134) to determine the location of distal reference feature (134) relative to position sensor (108).

Given that predetermined reference distance (136) and predetermined longitudinal sensor distance (144) are constants in the present example, the reference table is unique to these particular distances (136, 144) and may be simplified to the correlations for use as discussed above. In addition, alternative reference tables unique to alternative predetermined distances (136, 144) for alternative cutting members and/or alternative reference features may be similarly stored in memory (not shown) for use by processor (12). By way of example, the operator may enter such alternative cutting members and/or alternative reference features into the memory (not shown) so that processor (12) may identify a proper reference table for use from among a plurality of alternative reference tables stored in memory (not shown). In another example, the operator may input discrete values for predetermine reference distance (136) and/or predetermined longitudinal sensor distance (144) into memory (not shown), and processor (12) may calculate coordinates as discussed above to determine the location of distal reference feature (134) relative to position sensor (108). In any case, a wide variety of cutting members (106) and/or reference features (134, 138) with different predetermined distances (136, 144) may be interchangeably used with surgical system (8) in one or more examples.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical system, comprising: (a) a body assembly defining a longitudinal axis and configured to receive a rotatable cutting member having a distal reference feature thereon, wherein at least a portion of the body assembly is configured to be inserted into an anatomical passageway of a patient for rotatably driving a cutting member within the anatomical passageway of the patient; (b) a securement mechanism operatively connected to the body assembly and configured to releasably and longitudinally secure the cutting member relative to the at least the portion of the body assembly in an adjustable longitudinal position for selectively repositioning the distal reference feature of the cutting member relative to the body assembly; (c) a base position sensor secured on the body assembly and configured to be detected as a base position by a navigation system for determining the base position relative to the anatomical passageway of the patient; and (d) a cutting member sensor secured on the body assembly and positioned a predetermined longitudinal sensor distance from the base position sensor, wherein the cutting member sensor is configured to detect the adjustable longitudinal position of the cutting member relative to the body assembly along the longitudinal axis, wherein the cutting member sensor and the base position sensor are configured to communicate with the navigation system for determining a position of the distal reference feature within the anatomical passageway of the patient in any selected adjustable longitudinal position of the cutting member.

### Example 2

The surgical system of Example 1, further comprising a cutting member received by the body assembly and configured to be rotatably driven within the anatomical passageway of the patient, wherein the cutting member includes a distal reference feature and a proximal reference feature, and wherein the proximal reference feature is configured to be sensed by the cutting member sensor to detect the adjustable longitudinal position of the cutting member relative to the body assembly along the longitudinal axis.

### Example 3

The surgical system of Example 2, wherein the distal reference feature is positioned a predetermined reference distance from the proximal reference feature along the longitudinal axis for determining the position of the distal reference feature within the anatomical passageway of the patient in any selected adjustable longitudinal position of the cutting member.

### Example 4

The surgical system of any one or more of Examples 2 through 3, wherein the cutting member includes a shaft extending distally toward a distal tip, and wherein the distal reference feature is the distal tip of the cutting member.

### Example 5

The surgical system of Example 4, wherein the cutting member further includes a proximal end portion positioned longitudinally opposite from the distal tip and having the shaft extending between the proximal shaft end and the distal tip, and wherein the proximal reference feature is the proximal end portion of the cutting member.

### Example 6

The surgical system of any one or more of Examples 2 through 3, wherein the cutting member includes a cutting feature configured to remove a tissue of the patient and a shaft extending distally toward a distal tip, and wherein the distal reference feature is at least a portion of the cutting feature.

### Example 7

The surgical system of any one or more of Examples 1 through 6, further comprising a navigation system configured to communicate with the base position sensor and the cutting member sensor, and wherein the navigation system is configured to determine the position of the distal reference feature within the anatomical passageway of the patient in any selected adjustable longitudinal position of the cutting member based on a detected base position of the base position sensor, a detected adjustable longitudinal position of the cutting member, and the predetermined longitudinal sensor distance.

### Example 8

The surgical system of any one or more of Examples 1 through 7, wherein the body assembly further includes a sleeve configured to receive the cutting member therein, and wherein the cutting member sensor and the base position sensor are each secured to the sleeve.

### Example 9

The surgical system of Example 8, wherein the sleeve distally extends along the longitudinal axis to a distal sleeve end portion, wherein the base position sensor is secured within the distal sleeve end portion.

### Example 10

The surgical system of any one or more of Examples 8 through 9, wherein the cutting member sensor is proximally positioned on the sleeve relative to the base position sensor, and wherein the predetermined longitudinal sensor distance is longitudinally defined from the cutting member sensor to the base position sensor.

### Example 11

The surgical system of any one or more of Examples 8 through 10, wherein the sleeve defines a hollow extending along the longitudinal axis and configured to receive the cutting member therein, and wherein the cutting member sensor surrounds the hollow.

### Example 12

The surgical system of any one or more of Examples 1 through 11, wherein the cutting member sensor includes a hollow core magnetic coil.

### Example 13

The surgical system of any one or more of Examples 1 through 12, wherein the body assembly further includes a handle configured to be gripped by an operator and a drive assembly operatively connected to the handle and configured to connect to the cutting member for rotatably driving the cutting member.

### Example 14

The surgical system of Example 1, further comprising a cutting member received by the body assembly and configured to be rotatably driven within the anatomical passageway of the patient, wherein the cutting member includes a distal reference feature and a proximal reference feature, wherein the proximal reference feature is configured to be sensed by the cutting member sensor to detect the adjustable longitudinal position of the cutting member relative to the body assembly along the longitudinal axis, wherein the distal reference feature is positioned a predetermined reference distance from the proximal reference feature along the longitudinal axis for determining the position of the distal reference feature within the anatomical passageway of the patient in any selected adjustable longitudinal position of the cutting member.

### Example 15

The surgical system of Example 14, further comprising a navigation system configured to communicate with the base position sensor and the cutting member sensor, and wherein the navigation system is configured to determine the position of the distal reference feature within the anatomical passageway of the patient in any selected adjustable longitudinal position of the cutting member based on the detected base position of the base position sensor, the detected adjustable longitudinal position of the cutting member, the predetermined longitudinal sensor distance, and a predetermined reference distance.

### Example 16

A surgical system comprising: (a) a cutting member configured to be rotatably driven and inserted into an anatomical passageway of a patient, the cutting member including: (i) a cutting feature configured to remove a tissue of a patient, (ii) a proximal reference feature, and (iii) a distal tip, wherein the distal tip is positioned a predetermined reference distance from the proximal reference feature; (b) a body assembly defining a longitudinal axis and configured to receive the cutting member along the longitudinal axis; (c) a securement mechanism operatively connected to the body assembly and configured to releasably and longitudinally secure the cutting member relative to the body assembly in an adjustable longitudinal position for selectively repositioning the distal tip of the cutting member relative to the body assembly; (d) a navigation system operatively connected to the body assembly; (e) a base position sensor secured on the body assembly and configured to be detected as a base position by the navigation system for determining the base position relative to the anatomical passageway of the patient; and (f) a cutting member sensor secured on the body assembly and positioned a predetermined longitudinal sensor distance from the base position sensor, wherein the cutting member sensor is configured to detect the adjustable longitudinal position of the cutting member relative to the body assembly along the longitudinal axis, wherein the navigation system is configured to determine a position of the distal tip within the anatomical passageway of the patient in any selected adjustable longitudinal position of the cutting member based on the detected base position of the base position sensor, the detected adjustable longitudinal position of the cutting member, the predetermined longitudinal sensor distance, and the predetermined reference distance.

### Example 17

The surgical system of Example 16, wherein the cutting member further includes a proximal end portion positioned longitudinally opposite from the distal tip, and wherein the proximal reference feature is the proximal end portion of the cutting member.

### Example 18

The surgical system of any one or more of Examples 16 through 17, wherein the body assembly further includes a sleeve configured to receive the cutting member therein, and wherein the cutting member sensor and the base position sensor are each secured to the sleeve.

### Example 19

A method of determining a position of a distal reference feature of a surgical system within an anatomical passageway of a patient, the surgical system including a body assembly defining a longitudinal axis, a cutting member distally extending from the body assembly along the longitudinal axis and including a distal reference feature and an oppositely positioned proximal reference feature, wherein the distal reference feature is positioned a predetermined reference distance from a proximal reference feature along the longitudinal axis, a navigation system operatively connected to the body assembly, a base position sensor secured on the body assembly and configured to be detected as a base position by the navigation system, and a cutting member sensor secured on the body assembly and positioned a predetermined longitudinal sensor distance from the base position sensor, the method comprising: (a) detecting the base position sensor with the navigation system to thereby determine the base position within the anatomical passageway; (b) sensing the proximal reference feature with the cutting member sensor to thereby detect an adjustable longitudinal position of the cutting member relative to the body assembly along the longitudinal axis; and (c) determining the position of the distal reference feature within the anatomical passageway of the patient based on the detected base position of the base position sensor, the detected adjustable longitudinal position of the cutting member, the predetermined reference distance, and the predetermined longitudinal sensor distance, and the predetermined reference distance.

### Example 20

The method of Example 19, wherein the distal reference feature is a distal tip of the cutting member.

IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical system, comprising:
(a) a body assembly defining a longitudinal axis and configured to receive a rotatable cutting member having a distal reference feature thereon,
wherein at least a portion of the body assembly is configured to be inserted into an anatomical passageway of a patient for rotatably driving a cutting member within the anatomical passageway of the patient;
(b) a securement mechanism operatively connected to the body assembly and configured to releasably and longitudinally secure the cutting member relative to the at least the portion of the body assembly in an adjustable longitudinal position for selectively repositioning the distal reference feature of the cutting member relative to the body assembly;
(c) a base position sensor secured on the body assembly and configured to be detected as a base position by a navigation system for determining the base position relative to the anatomical passageway of the patient; and
(d) a cutting member sensor secured on the body assembly and positioned a predetermined longitudinal sensor distance from the base position sensor, wherein the cutting member sensor is configured to detect the adjustable longitudinal position of the cutting member relative to the body assembly along the longitudinal axis,
wherein the cutting member sensor and the base position sensor are configured to communicate with the navigation system for determining a position of the distal reference feature within the anatomical passageway of the patient in any selected adjustable longitudinal position of the cutting member.

2. The surgical system of claim 1, further comprising a cutting member received by the body assembly and configured to be rotatably driven within the anatomical passageway of the patient, wherein the cutting member includes a distal reference feature and a proximal reference feature, and wherein the proximal reference feature is configured to be sensed by the cutting member sensor to detect the adjustable longitudinal position of the cutting member relative to the body assembly along the longitudinal axis.

3. The surgical system of claim 2, wherein the distal reference feature is positioned a predetermined reference distance from the proximal reference feature along the longitudinal axis for determining the position of the distal reference feature within the anatomical passageway of the patient in any selected adjustable longitudinal position of the cutting member.

4. The surgical system of claim 2, wherein the cutting member includes a shaft extending distally toward a distal tip, and wherein the distal reference feature is the distal tip of the cutting member.

5. The surgical system of claim 4, wherein the cutting member further includes a proximal end portion positioned longitudinally opposite from the distal tip and having the shaft extending between the proximal shaft end and the distal tip, and wherein the proximal reference feature is the proximal end portion of the cutting member.

6. The surgical system of claim 2, wherein the cutting member includes a cutting feature configured to remove a tissue of the patient and a shaft extending distally toward a distal tip, and wherein the distal reference feature is at least a portion of the cutting feature.

7. The surgical system of claim 1, further comprising a navigation system configured to communicate with the base position sensor and the cutting member sensor, and wherein the navigation system is configured to determine the position of the distal reference feature within the anatomical passageway of the patient in any selected adjustable longitudinal position of the cutting member based on a detected base position of the base position sensor, a detected adjustable longitudinal position of the cutting member, and the predetermined longitudinal sensor distance.

8. The surgical system of claim 1, wherein the body assembly further includes a sleeve configured to receive the cutting member therein, and wherein the cutting member sensor and the base position sensor are each secured to the sleeve.

9. The surgical system of claim 8, wherein the sleeve distally extends along the longitudinal axis to a distal sleeve end portion, wherein the base position sensor is secured within the distal sleeve end portion.

10. The surgical system of claim 8, wherein the cutting member sensor is proximally positioned on the sleeve relative to the base position sensor, and wherein the predetermined longitudinal sensor distance is longitudinally defined from the cutting member sensor to the base position sensor.

11. The surgical system of claim 8, wherein the sleeve defines a hollow extending along the longitudinal axis and configured to receive the cutting member therein, and wherein the cutting member sensor surrounds the hollow.

12. The surgical system of claim 1, wherein the cutting member sensor includes a hollow core magnetic coil.

13. The surgical system of claim 1, wherein the body assembly further includes a handle configured to be gripped by an operator and a drive assembly operatively connected to the handle and configured to connect to the cutting member for rotatably driving the cutting member.

14. The surgical system of claim 1, further comprising a cutting member received by the body assembly and configured to be rotatably driven within the anatomical passageway of the patient, wherein the cutting member includes a distal reference feature and a proximal reference feature, wherein the proximal reference feature is configured to be sensed by the cutting member sensor to detect the adjustable longitudinal position of the cutting member relative to the body assembly along the longitudinal axis, wherein the distal reference feature is positioned a predetermined reference distance from the proximal reference feature along the longitudinal axis for determining the position of the distal reference feature within the anatomical passageway of the patient in any selected adjustable longitudinal position of the cutting member.

15. The surgical system of claim 14, further comprising a navigation system configured to communicate with the base position sensor and the cutting member sensor, and wherein the navigation system is configured to determine the position of the distal reference feature within the anatomical passageway of the patient in any selected adjustable longitudinal position of the cutting member based on the detected base position of the base position sensor, the detected adjustable longitudinal position of the cutting member, the predetermined longitudinal sensor distance, and a predetermined reference distance.

16. A surgical system comprising:
(a) a cutting member configured to be rotatably driven and inserted into an anatomical passageway of a patient, the cutting member including:
(i) a cutting feature configured to remove a tissue of a patient,
(ii) a proximal reference feature, and
(iii) a distal tip, wherein the distal tip is positioned a predetermined reference distance from the proximal reference feature;
(b) a body assembly defining a longitudinal axis and configured to receive the cutting member along the longitudinal axis;
(c) a securement mechanism operatively connected to the body assembly and configured to releasably and longitudinally secure the cutting member relative to the body assembly in an adjustable longitudinal position for selectively repositioning the distal tip of the cutting member relative to the body assembly;
(d) a navigation system operatively connected to the body assembly;
(e) a base position sensor secured on the body assembly and configured to be detected as a base position by the navigation system for determining the base position relative to the anatomical passageway of the patient; and
(f) a cutting member sensor secured on the body assembly and positioned a predetermined longitudinal sensor distance from the base position sensor, wherein the cutting member sensor is configured to detect the adjustable longitudinal position of the cutting member relative to the body assembly along the longitudinal axis,
wherein the navigation system is configured to determine a position of the distal tip within the anatomical passageway of the patient in any selected adjustable longitudinal position of the cutting member based on the detected base position of the base position sensor, the detected adjustable longitudinal position of the cutting member, the predetermined longitudinal sensor distance, and the predetermined reference distance.

17. The surgical system of claim 16, wherein the cutting member further includes a proximal end portion positioned longitudinally opposite from the distal tip, and wherein the proximal reference feature is the proximal end portion of the cutting member.

18. The surgical system of claim 16, wherein the body assembly further includes a sleeve configured to receive the cutting member therein, and wherein the cutting member sensor and the base position sensor are each secured to the sleeve.

19. A method of determining a position of a distal reference feature of a surgical system within an anatomical passageway of a patient, the surgical system including a body assembly defining a longitudinal axis, a cutting member distally extending from the body assembly along the longitudinal axis and including a distal reference feature and an oppositely positioned proximal reference feature, wherein the distal reference feature is positioned a predetermined reference distance from a proximal reference feature along the longitudinal axis, a navigation system operatively connected to the body assembly, a base position sensor secured on the body assembly and configured to be detected as a base position by the navigation system, and a cutting member sensor secured on the body assembly and positioned a predetermined longitudinal sensor distance from the base position sensor, the method comprising:
(a) detecting the base position sensor with the navigation system to thereby determine the base position within the anatomical passageway;
(b) sensing the proximal reference feature with the cutting member sensor to thereby detect an adjustable longitudinal position of the cutting member relative to the body assembly along the longitudinal axis; and
(c) determining the position of the distal reference feature within the anatomical passageway of the patient based on the detected base position of the base position sensor, the detected adjustable longitudinal position of the cutting member, the predetermined reference distance, and the predetermined longitudinal sensor distance, and the predetermined reference distance.

20. The method of claim 19, wherein the distal reference feature is a distal tip of the cutting member.
